(19) United States
(12) Patent Application Publication
(10) Pub. No.: US 2018/0344945 A1
(43) Pub. Date: Dec. 6, 2018

(54) INJECTION DEVICE

(71) Applicant: Sanofi-Aventis GmbH, Frankfurt am Main (DE)

(72) Inventors: Stefan WENDLAND, Frankfurt am Main (DE); Michael HARMS, Frankfurt am Main (DE)

(21) Appl. No.: 15/778,385

(22) PCT Filed: Nov. 21, 2016
(86) PCT No.: PCT/EP2016/078251

§ 371 Date: May 23, 2018

(30) Foreign Application Priority Data
Nov. 27, 2015 (EP) 15196680.1

(51) Int. Cl. A61M 5/32; A61M 5/20

(52) U.S. Cl. CPC A61M 5/3204; A61M 5/2046

(57) ABSTRACT

The present disclosure relates to an injection device. The injection device comprises a cap and a body for holding a syringe that has a needle at one end. The cap is removably attached to the body and comprises a needle shield to cover said needle. The injection device further comprises a configuration adapted to increase the gas pressure at an interface of the injection device that is disposed between the body and a portion of the cap to urge the body and said portion of the cap apart.

### CROSS REFERENCE TO RELATED APPLICATIONS

The present application is the national stage entry of International Patent Application No. PCT/EP2016/078251, filed on Nov. 21, 2016, and claims priority to Application No. EP 15196680.1, filed in on Nov. 27, 2015, the disclosures of which are expressly incorporated herein in entirety by reference thereto.

### TECHNICAL FIELD

The present disclosure relates to an injection device.

### BACKGROUND

Injection devices, such as auto-injectors, are known in the art for dispensing a medicament to the injection site of a patient. Such injection devices typically comprise a body and a cap. A needle syringe is located in the body. The cap is removably attached to the body to shield the needle of the needle syringe. To dispense the medicament, the cap is first removed from the body to expose the needle. The needle is then inserted into the body of the patient at the injection site to dispense the medicament.

It is important that the cap is held onto the body with sufficient force to ensure that the cap is not accidentally removed from the body during transport and storage of the injection device. This ensures that the needle is kept sterile and also prevents the sharp needle from causing injury. However, the force required to hold the cap and body together can make it difficult for the patient to intentionally remove the cap from the body prior to injection, particularly if the patient is elderly or infirm.

### SUMMARY

In certain aspects, an improved injection device is provided.

In certain aspects, there is provided an injection device comprising: a body for holding a syringe that has a needle at one end; a cap that is removably attached to the body and comprises a needle shield to cover said needle; and, a configuration adapted to increase the gas pressure at an interface of the injection device that is disposed between the body and a portion of the cap to urge the body and said portion of the cap apart.

The patient can operate said configuration adapted to increase the gas pressure to urge said portion of the cap away from the body to facilitate removal of the cap from the body.

In one embodiment, the interface comprises a chamber that is disposed between the body and said portion of the cap when the cap is attached to the body. The chamber may be disposed between said syringe and said portion of the cap when the cap is attached to the body and a syringe is received in the body. Therefore, the patient can operate said configuration to increase the gas pressure in the chamber such that the body and said portion of the cap are urged apart.

In one embodiment, the interface is disposed between a surface of the cap and a surface of the body or syringe. The surfaces may be opposing surfaces. The chamber may be disposed between the surfaces.

The needle shield may comprise said portion of the cap. Therefore, the needle shield is urged away from the body when the gas pressure at the interface is increased.

In one embodiment, the configuration adapted to increase the gas pressure comprises a compressed gas source that is configured to be selectively fluidly communicated with the interface. Therefore, when the compressed gas source is fluidly communicated with the interface said portion of the cap is urged away from the body to facilitate easy removal of the cap from the body.

The compressed gas source may be disposed in the body or cap. This improves the portability of the injector device. In one embodiment, the compressed gas source is selectively fluidly communicated with the interface via a filter. The filter removes contaminants from gas flowing to the interface. In one embodiment, the injection device comprises a valve that is operable to communicate the compressed gas source with the interface.

The compressed gas source may be selectively fluidly communicated with the interface by one or more conduits. In one embodiment, a first conduit is provided in the body and is fluidly connected to the compressed gas source and a second conduit is provided in the cap and is fluidly connected to the interface. The first and second conduits may be fluidly connected when the cap is attached to the body. The first and second conduits may be separated when the cap is removed from the body.

In one embodiment, the injector device further comprises a base station that has a housing with a recess to receive the cap. The compressed gas source may be disposed in the base station.

The injector device may conveniently be held in the base station prior to injection. The base station may be configured such that the interface is selectively fluidly communicated with the compressed gas source when the cap is received in the recess.

In one embodiment, the base station comprises a cap collection space configured such that when the cap is received in the recess of the housing and the configuration adapted to increase the gas pressure urges said portion of the cap away from the body, the cap moves into the cap collection space. This allows for easy collection of the cap. After the cap has moved into the collection space, it may be retained in the collection space until removal by the user. Alternatively, after the cap has moved into the collection space it is ejected from the base station for collection by the user.

The configuration adapted to increase the gas pressure may comprise a space that is fluidly communicated with the interface and is compressible to increase the gas pressure at the interface. Therefore, the patient is able to manually increase the gas pressure at the interface without requiring a supply of compressed gas to urge the body and said portion of the cap apart. The space may be disposed in the cap.

In one embodiment, at least a part of the cap is deformable to compress the space. Therefore, the patient is able to deform said part of the cap to urge said portion of the cap away from the body. In an alternative embodiment, at least a part of the body is deformable to compress the space. In yet another embodiment, the injection device further comprises a base station and at least a part of the base station is deformable to compress the space.

In an alternative embodiment, the cap comprises an actuator that is slidable relative to the body to compress the space. Therefore, the patient is able to slide the actuator relative to the body to urge said portion of the cap away from the body. The actuator may comprise an end cap that is slidably received in the needle shield. Alternatively, the body comprises a slidable actuator. The actuator may comprise an annular peripheral wall. An end wall may be provided at an end of the peripheral wall and may be arranged such that the patient is able to exert a force on the end wall to slide the actuator relative to the body.

In one embodiment, the injector device is configured such that a force exerted on the actuator to slide the actuator relative to the body to compress the space results in a larger force being exerted to urge the body and said portion of the cap apart. This reduces the force that must be exerted by the patient to remove the cap from the body.

The injection device may comprise a syringe that has a needle at one end and is received in the body. The needle shield may be in frictional engagement with the syringe. The syringe may contain a medicament.

In one embodiment, the injection device is an auto-injector.

In certain aspects, there is provided a method of removing a cap from a body of an injection device, comprising increasing the gas pressure at an interface of the injection device that is disposed between the body and a portion of the cap to urge the body and said portion of the cap apart. The injection device may comprise one or more of the features of the injection device described hereinbefore.

These and other aspects of the invention will be apparent from and elucidated with reference to the embodiments described hereinafter.

### BRIEF DESCRIPTION OF THE FIGURES

Embodiments of the invention will now be described, by way of example only, with reference to the accompanying drawings, in which:

FIG. 1A is a schematic side view of an auto-injector illustrative of some embodiments, with a cap attached to a body of the injection device;

FIG. 1B is a schematic side view of the auto-injector of FIG. 1A, with the cap removed from the body;

FIG. 2 is a schematic cross-sectional side view of part of an auto-injector according to a first embodiment of the invention, wherein an actuator is in a first position;

FIG. 3 is a schematic cross-sectional side view of part of the auto-injector of FIG. 2, wherein the actuator is in a second position;

FIG. 4 is a schematic cross-sectional side view of part of the auto-injector of FIG. 2, wherein the actuator is in a third position;

FIG. 5 is a schematic cross-sectional side view of part of an auto-injector according to a second embodiment of the invention, wherein a cap is attached to a body of the auto-injector and a valve is in a closed state;

FIG. 6 is schematic cross-sectional side view of part of the body of the auto-injector of FIG. 5;

FIG. 7 is a schematic cross-sectional side view of the auto-injector of FIG. 5, wherein the cap is attached to the body and the valve is in an open state;

FIG. 8 is a schematic cross-sectional side view of the auto-injector of FIG. 5, wherein the cap is removed from the body;

FIG. 9 is a schematic cross-sectional side view of part of an auto-injector according to a third embodiment of the invention, wherein a cap is attached to a body of the auto-injector and a valve is in a closed state;

FIG. 10 is a schematic cross-sectional side view of the auto-injector of FIG. 9, wherein the cap is attached to the body and the valve is in an open state;

FIG. 11 is a schematic cross-sectional side view of the auto-injector of FIG. 9, wherein the cap is removed from the body;

FIG. 12 is a schematic cross-sectional side view of part of an auto-injector according to a fourth embodiment of the invention, wherein a cap and body of the auto-injector are spaced from a base station;

FIG. 13 is a schematic cross-sectional side view of the auto-injector of FIG. 12, wherein the cap and body of the auto-injector are received in a recess of the base station and a valve is in a closed state;

FIG. 14 is a schematic cross-sectional side view of the auto-injector of FIG. 12, wherein the cap and body of the auto-injector are received in the recess of the base station and the valve is in an open state;

FIG. 15 is a schematic cross-sectional side view of the auto-injector of FIG. 12, wherein the cap and body of the auto-injector are received in the recess of the base station and the cap is separated from the body; and,

FIG. 16 is a schematic cross-sectional side view of the auto-injector of FIG. 12, wherein the cap and body of the auto-injector are removed from the recess of the base station and the cap is separated from the body.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

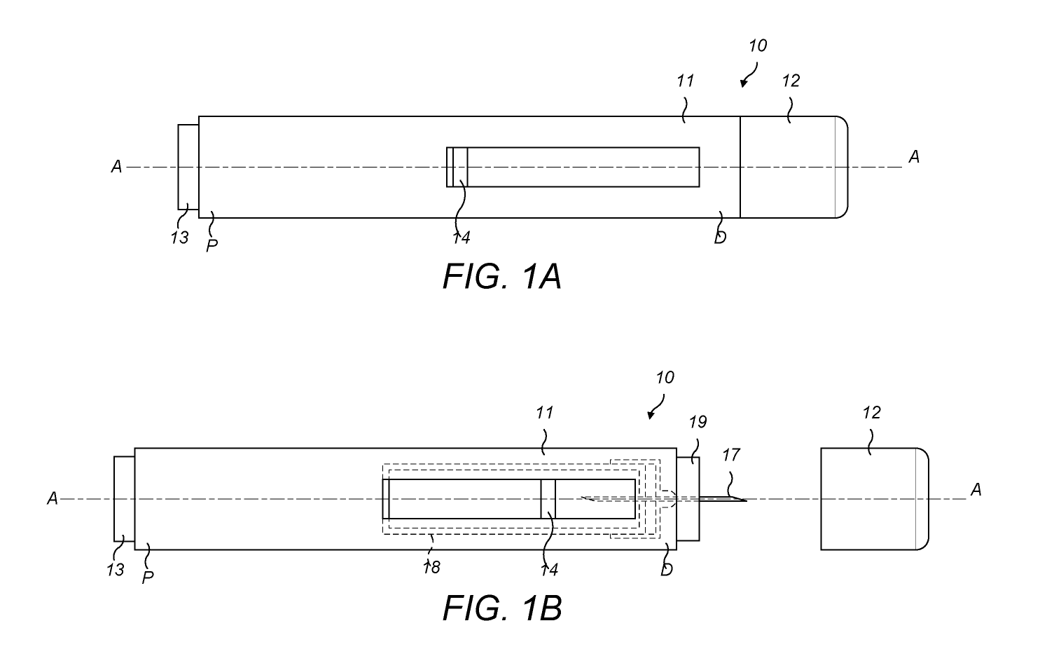

According to some embodiments of the present disclosure, an exemplary drug delivery device 10 is shown in FIGS. 1A & 1B. Device 10, as described above, is configured to inject a medicament into a patient's body. Device 10 includes a housing 11 which typically contains a reservoir containing the medicament to be injected (e.g., a syringe) and the components required to facilitate one or more steps of the delivery process. Device 10 can also include a cap assembly 12 that can be detachably mounted to the housing 11. Typically a user must remove cap 12 from housing 11 before device 10 can be operated.

As shown, housing 11 is substantially cylindrical and has a substantially constant diameter along the longitudinal axis A-A. The housing 11 has a distal region D and a proximal region P. The term “distal” refers to a location that is relatively closer to a site of injection, and the term “proximal” refers to a location that is relatively further away from the injection site.

Device 10 can also include a needle sleeve 19 coupled to housing 11 to permit movement of sleeve 19 relative to housing 11. For example, sleeve 19 can move in a longitudinal direction parallel to longitudinal axis A-A. Specifically, movement of sleeve 19 in a proximal direction can permit a needle 17 to extend from distal region D of housing 11.

Insertion of needle 17 can occur via several mechanisms. For example, needle 17 may be fixedly located relative to housing 11 and initially be located within an extended needle sleeve 19. Proximal movement of sleeve 19 by placing a distal end of sleeve 19 against a patient's body and moving housing 11 in a distal direction will uncover the distal end of needle 17. Such relative movement allows the distal end of needle 17 to extend into the patient's body. Such insertion is termed “manual” insertion as needle 17 is manually inserted via the patient's manual movement of housing 11 relative to sleeve 19.

Another form of insertion is “automated,” whereby needle 17 moves relative to housing 11. Such insertion can be triggered by movement of sleeve 19 or by another form of activation, such as, for example, a button 13. As shown in FIGS. 1A & 1B, button 13 is located at a proximal end of housing 11. However, in other embodiments, button 13 could be located on a side of housing 11.

Other manual or automated features can include drug injection or needle retraction, or both. Injection is the process by which a bung or piston 14 is moved from a proximal location within a syringe 18 to a more distal location within the syringe 18 in order to force a medicament from the syringe 18 through needle 17. In some embodiments, a drive spring (not shown) is under compression before device 10 is activated. A proximal end of the drive spring can be fixed within proximal region P of housing 11, and a distal end of the drive spring can be configured to apply a compressive force to a proximal surface of piston 14. Following activation, at least part of the energy stored in the drive spring can be applied to the proximal surface of piston 14. This compressive force can act on piston 14 to move it in a distal direction. Such distal movement acts to compress the liquid medicament within the syringe 18, forcing it out of needle 17.

Following injection, needle 17 can be retracted within sleeve 19 or housing 11. Retraction can occur when sleeve 19 moves distally as a user removes device 10 from a patient's body. This can occur as needle 17 remains fixedly located relative to housing 11. Once a distal end of sleeve 19 has moved past a distal end of needle 17, and needle 17 is covered, sleeve 19 can be locked. Such locking can include locking any proximal movement of sleeve 19 relative to housing 11.

Another form of needle retraction can occur if needle 17 is moved relative to housing 11. Such movement can occur if the syringe 18 within housing 11 is moved in a proximal direction relative to housing 11. This proximal movement can be achieved by using a retraction spring (not shown), located in distal region D. A compressed retraction spring, when activated, can supply sufficient force to the syringe 18 to move it in a proximal direction. Following sufficient retraction, any relative movement between needle 17 and housing 11 can be locked with a locking mechanism. In addition, button 13 or other components of device 10 can be locked as required.

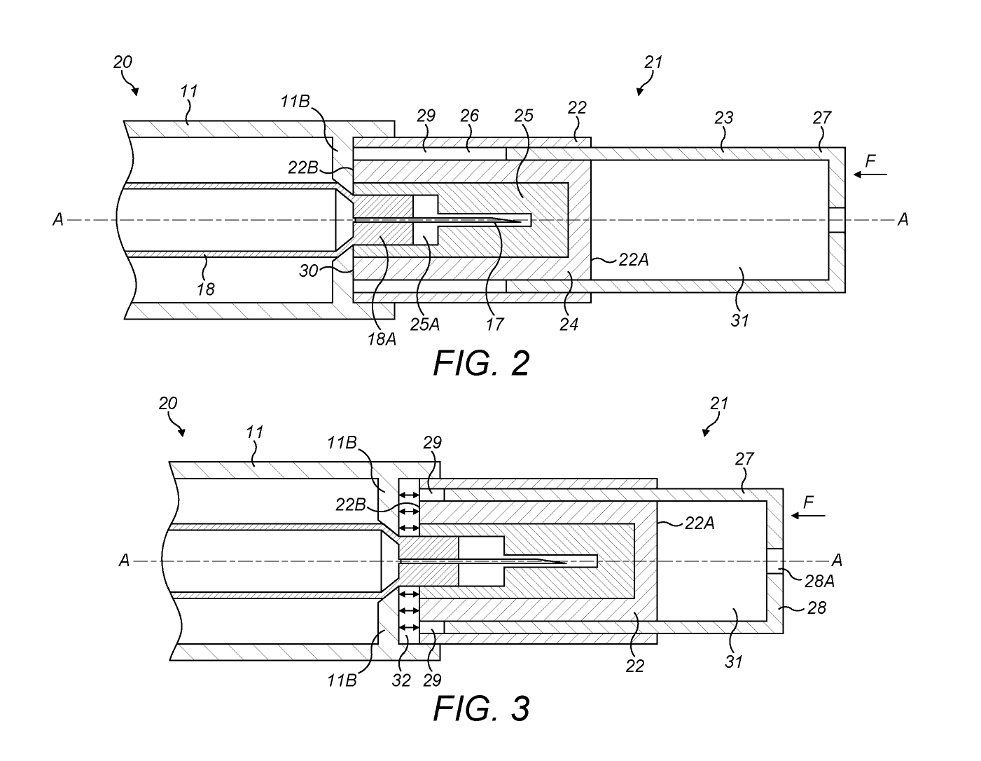

Referring now to FIGS. 2 to 4, part of an injection device 20 according to a first embodiment of the invention is shown. The injection device 20 is in the form of an auto-injector 20 that has similar features to the auto-injector 10 described above in relation to FIGS. 1A and 1B, with like features retaining the same reference numerals. A difference is that the cap 12 of the auto-injector 10 described above is omitted and is replaced with an alternative cap 21.

The cap 21 of the auto-injector 20 of the first embodiment of the invention comprises a needle shield 22 and an actuator 23. The needle shield 22 comprises a housing 24 and an inner sheath 25. The inner sheath 25 is fixedly secured in the housing 24. The inner sheath 25 comprises a cylindrical recess 25A. The recess 25A is configured to receive an end portion 18A of the syringe 18 such that the needle 17 is shielded by the inner sheath 25. The friction between the inner sheath 25 and the end portion 18A of the syringe 18 is sufficient to hold the needle shield 22 in place covering the needle 17.

The housing 24 of the needle shield 22 comprises an annular recess 26 that extends through the needle shield 22 from the distal end 22A to the proximal end 22B of the needle shield 22.

The actuator 23 is in the form of an end cap 23 that comprises a cylindrical peripheral wall 27 and an end wall 28. The peripheral wall 27 of the end cap 23 is slidably received in the annular recess 26. The end cap 23 is concentrically aligned with the needle shield 22. The portion of the annular recess 26 that is disposed between the end cap 23 and the proximal end 22B of the needle shield 22 comprises a space 29.

The peripheral wall 27 of the end cap 23 has a similar cross-sectional shape to the annular recess 26 in the housing 24 of the needle shield 22 such that the peripheral wall 27 fits snugly in the annular recess 26 to seal the space 29 at the peripheral end 22A of the needle shield 22.

The proximal end 22B of the needle shield 22 comprises a support strut (not shown) that transverses the annular recess 26 to connect the portions of the housing 24 on either side of the annular recess 26. The peripheral wall 27 of the end cap 23 comprises a slot (not shown) that slidably receives said strut. In another embodiment (not shown), the distal end 22A of the needle shield 22 comprises the support strut. Alternatively, the support strut may be located between the distal and proximal ends 22A, 22B of the needle shield 22.

The body 11 comprises an open distal end 11A and an annular lip 11B that is disposed near to the open distal end 11A. The annular lip 11B extends radially inwardly and is sealed against the syringe 18.

The cap 21 is initially attached to the body 11 such that the end portion 18A of the syringe 18 is received in the recess 25A of the inner sheath 25 and the proximal end 22B of the needle shield 22 is adjacent to the lip 11B of the body 11 (as shown in FIG. 2). Thus, the needle 17 is covered by the needle shield 22 to keep the needle 17 sterile and to prevent the needle 17 from causing injury to the patient. When the cap 21 is initially attached to the body 11, the end cap 23 is in a first position (as shown in FIG. 2) wherein the end wall 28 of the end cap 23 is axially spaced from the distal end 22A of the needle shield 22 by a gap 31. Optionally, the end cap 23 may be held in the first position by a lock, which is configured to prevent axial movement of the end cap 23 relative to the needle shield 22. To remove the cap 21 from the body 11, the user first unlocks the lock such that the end cap 23 can be moved away from the first position. The lock may comprise, for example, a bayonet connection. The bayonet connection is released to unlock the lock. The lock may alternatively comprise a screw thread between the end cap 23 and needle shield 22. Thus, the end cap 23 is first twisted relative to the needle shield 22 to unlock the lock.

The lip 11B of the body 11 and a portion of the needle shield 22 at the proximal end 22B of the needle shield 22 comprise an interface 30 between the cap 21 and the body 11. More specifically, the distally facing surface of the lip 11B and the opposing surface of the needle shield 22 at the proximal end 22B thereof comprise said interface 30 between the cap 21 and the body 11.

The periphery of the housing 24 of the needle shield 22 is in sealing engagement with the peripheral wall of the body 11 when the cap 21 is attached to the body 11 such that the interface 30 is sealed from atmosphere.

When the cap 21 is attached to the body 11 and the end cap 23 is moved axially towards the body 11 from the first position (in the direction of arrow ‘F’ shown in FIGS. 2 and 3), the peripheral wall 27 of the end cap 23 slides in the annular recess 26 of the housing 24 to reduce the volume of the space 29 in the needle shield 22. This causes the air in the space 29 to be compressed such that the air pressure in the space 29 is increased.

The space 29 in the needle shield 22 is fluidly communicated with the interface 30. Therefore, when the end cap 23 is moved axially towards the body 11 to increase the air pressure in the space 29, the air pressure at the interface 30 also increases. The increased air pressure at the interface 30 exerts a force on the lip 11B of the body 11 such that the needle shield 22 and body 11 are urged apart.

The end wall 28 of the end cap 23 comprises an aperture 28A that allows air to flow out of the gap 31 between the distal end 22A of the needle shield 22 and the end wall 28 of the end cap 23. This allows for the air pressure in the gap 31 to equalise with the atmosphere to facilitate movement of the end cap 23 relative to the needle shield 22.

To inject medicament, the cap 21 is first removed from the body 11 to expose the needle 17. Removal of the cap 21 from the body 11 is achieved by the patient exerting a force on the end cap 23 (in the direction of arrow ‘F’ in FIGS. 2 and 3) to urge the end cap 23 axially towards the body 11. This causes the end cap 23 to slide relative to the needle shield 22 such that the end cap 23 moves from the first position (shown in FIG. 2) to a second position (shown in FIG. 3).

When the end cap 23 is moved from the first position to the second position, the volume of the space 29 in the needle shield 22 is reduced and therefore the air pressure in the space 29 and at the interface 30 between the cap 21 and body 11 is increased. The increased air pressure at the interface 30 exerts a force on the lip 11B of the body 11 to urge the body 11 and needle shield 22 axially apart such that a chamber 32 is formed at the interface 30 between the lip 11B and the needle shield 22 (as shown in FIG. 3).

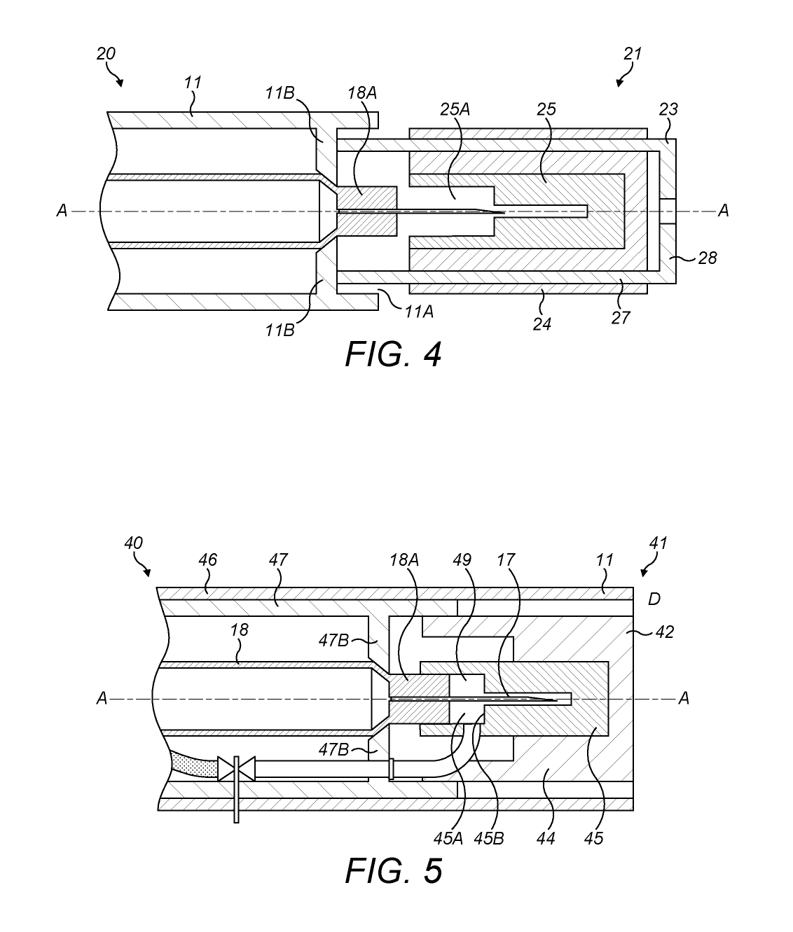

To remove the cap 21 from the body 11, the patient continues to urge the end cap 23 axially towards the body 11. This causes the end cap 23 to slide relative to the needle shield 22 such that the volume of the space 29 is further decreased. Therefore, the air pressure in the space 29 and in the chamber 32 at the interface 30 is increased to exert a force on the proximal end 22B of the needle shield 22 and the lip 11B of the body 11 such that the needle shield 22 and body 11 are urged further apart. The needle shield 22 continues to move away from the body 11 as the user pushes the end cap 23 towards the body 11 until the needle shield 22 is separated from the body 11 and the end cap 23 is moved to a third position (as shown in FIG. 4), wherein the peripheral wall 27 of the end cap 23 protrudes from the proximal end 22B of the needle shield 22.

The needle 17 is fixed relative to the body 11. Therefore, as the needle shield 22 is urged away from the body 11 due to the patient urging the end cap 23 from the first position to the third position, the needle shield 22 moves axially away from the needle 17 such that the inner sheath 25 is removed from the end portion 18A of the syringe 18. Once the end portion 18A of the syringe 18 has been fully removed from the recess 25A in the inner sheath 25, the friction between the cap 21 and the body 11 is reduced such that the cap 21 can easily be removed from the body 11 simply by pulling the end cap 23 away from the body 11 to expose the needle 17. The open distal end 11A of the body 11 is then pressed up against an injection site of the patient and the dispense button (not shown) is pressed to dispense medicament to the injection site.

The auto-injector 20 is configured such that a force exerted by the patient on the end cap 23 to urge the end cap 23 axially towards the body 11 from the first position results in a greater force being exerted on the body 11 relative to the needle shield 22 to urge the body 11 and needle shield 22 apart. This facilitates removal of the cap 21 from the body 11. This is achieved by dimensioning the space 29 relative to the interface 30 such that the surface area of the end of the peripheral wall 27 of the end cap 23, which acts on the air in the space 29 to compress the air, is smaller than the surface area of the body 11 that is acted on by the air at the interface 30, which is at the same pressure as the air in the space 29, when the needle shield 22 is spaced from the lip 11B of the body 11 to form a chamber 32 (as shown in FIG. 3). Therefore, when the needle shield 22 is spaced from the lip 11B of the body 11, axial movement of the end cap 23 towards the body 11 by a first distance results in axial movement of the needle shield 22 away from the body 11 by a second distance, which is smaller than the first distance. In the one embodiment, the cross-sectional area of the peripheral wall 27 of the end cap 23 is smaller than the cross-sectional area of the lip 11 B, when viewed in the sliding direction of the end cap 23.

The pushing movement of the end cap 23 relative to the body 11 to separate the needle shield 22 from the body 11 may be easier for the patient to perform than pulling or twisting motions, particularly if the patient is elderly or infirm.

Although in the above described embodiment the space 29 comprises air, in alternative embodiments (not shown) the space comprises another gas. Preferably the gas has a low compressibility. Preferably, the gas is non-toxic and/or inert. The gas may be, for example, carbon dioxide, argon or helium.

In the above described embodiment the auto-injector 20 comprises an actuator 23 that is slidable relative to the needle shield 22 to reduce the volume of the space 29 such that the air pressure in the space 29 is increased. Thus, the pressure at the interface 30 is increased. The force acting on the interface 30 is the product of the pressure in the space 29 and the area of the interface 30 and so when the pressure in the space 29 is increased the body 11 and needle shield 22 are urged apart. Therefore, the needle shield 22 and actuator 23 together form a configuration adapted to increase the gas pressure at the interface 30. However, in an alternative embodiment (not shown) the sliding actuator is omitted and is replaced by an alternative configuration adapted to increase the gas pressure at the interface 30. In one such embodiment (not shown), the cap comprises a flexible end cap which has a cavity that defines a deformable space. The deformable space is fluidly communicated with the interface and is sealed from atmosphere when the cap is attached to the body. The flexible end cap is squeezed by the patient to reduce the volume of the deformable space such that the air pressure at the interface is increased, causing the needle shield to be urged axially away from the body. In another such embodiment (not shown), the body comprises a flexible portion which has a cavity that defines a deformable space. The deformable space is fluidly communicated with the interface and is sealed from the atmosphere when the cap is attached to the body. The flexible portion of the body is squeezed to increase the pressure at the interface such that the needle shield is urged axially away from the body.

In the above described embodiment, the proximal end 22B of the needle shield 22 abuts the lip 11B of the body 11 when the end cap 23 is in the first position. However, in an alternative embodiment (not shown) the proximal end of the needle shield is axially spaced from the lip when the end cap is in the first position such that a small chamber is disposed between the needle shield and the lip. When the end cap is moved from the first position to the second position the gas pressure in the small chamber is increased such that the needle shield is urged away from the body.

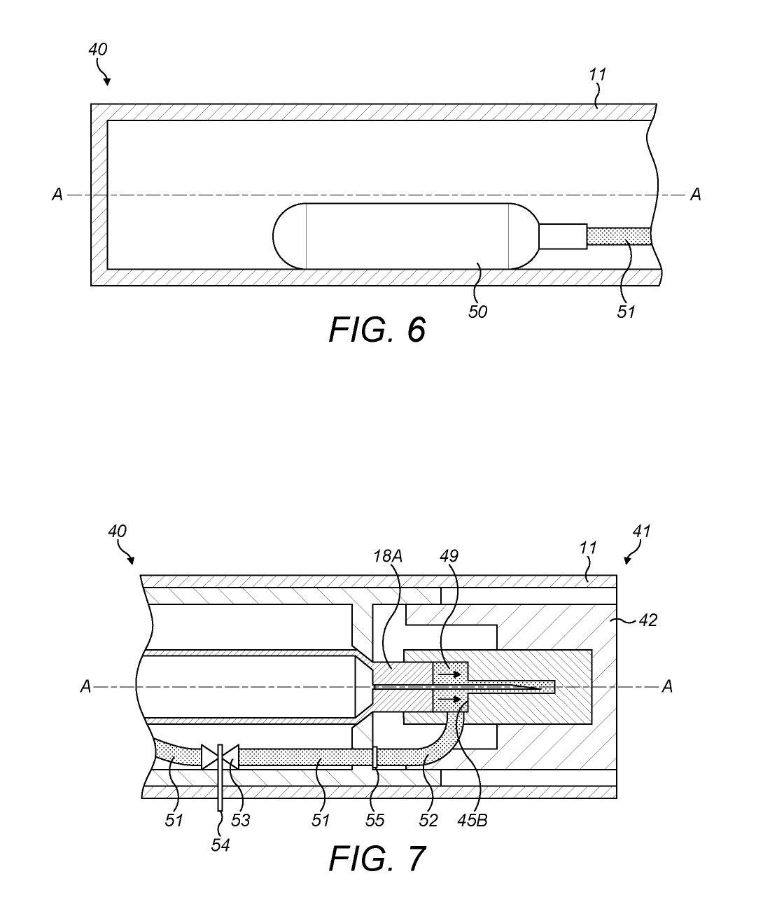

Referring now to FIGS. 5 to 8, part of an injection device 40 according to a second embodiment of the invention is shown. The injection device 40 is in the form of an auto-injector 40 that has similar features to the auto-injector 10 described above in relation to FIGS. 1A and 1B, with like features retaining the same reference numerals. A difference is that the cap 12 of the auto-injector 10 described above is omitted and is replaced with an alternative cap 41.

The cap 41 of the auto-injector 40 of the second embodiment of the invention comprises a needle shield 42. The needle shield 42 comprises a housing 44 and an inner sheath 45. The inner sheath 45 is fixedly secured in the housing 44. The inner sheath 45 comprises a cylindrical recess 45A.

The body 11 comprises an outer casing 46 and an inner sleeve 47 that is disposed in the outer casing 46. The inner sleeve 47 is fixed relative to the outer casing 46. The needle shield 42 is received in an open peripheral end 47A of the inner sleeve 47 when the cap 41 is attached to the body 11 such that the end portion 18A of the syringe 18 is received in the recess 45A of the inner sheath 45 (as shown in FIG. 5). Therefore, the needle 17 is shielded by the inner sheath 45. The friction between housing 44 and the inner sleeve 47 and between the inner sheath 45 and the end portion 18A of the syringe 18 is sufficient to hold the needle shield 42 in place covering the needle 17.

The inner sleeve 47 comprises a lip 47B that extends radially inwardly from a peripheral wall of the inner sleeve 47 to seal against the syringe 18.

The auto-injector 40 comprises an interface 48 that is disposed between the cap 41 and the body 11 when the cap 41 is attached to the body 11. The interface 48 comprises a chamber 49 that is disposed in the recess 45A of the inner sheath 45, between the end portion 18A of the syringe 18 and an internal surface 45B of the inner sheath 45 at the distal end of the recess 45A. The periphery of the end portion 18A of the syringe 18 seals against the inner sheath 45 when the cap 41 is attached to the body 11 such that the chamber 49 is sealed from atmosphere. The gas in the chamber 49 is initially at atmospheric pressure. Alternatively, the gas in the chamber 49 may initially be at a pressure that is below or above atmospheric pressure.

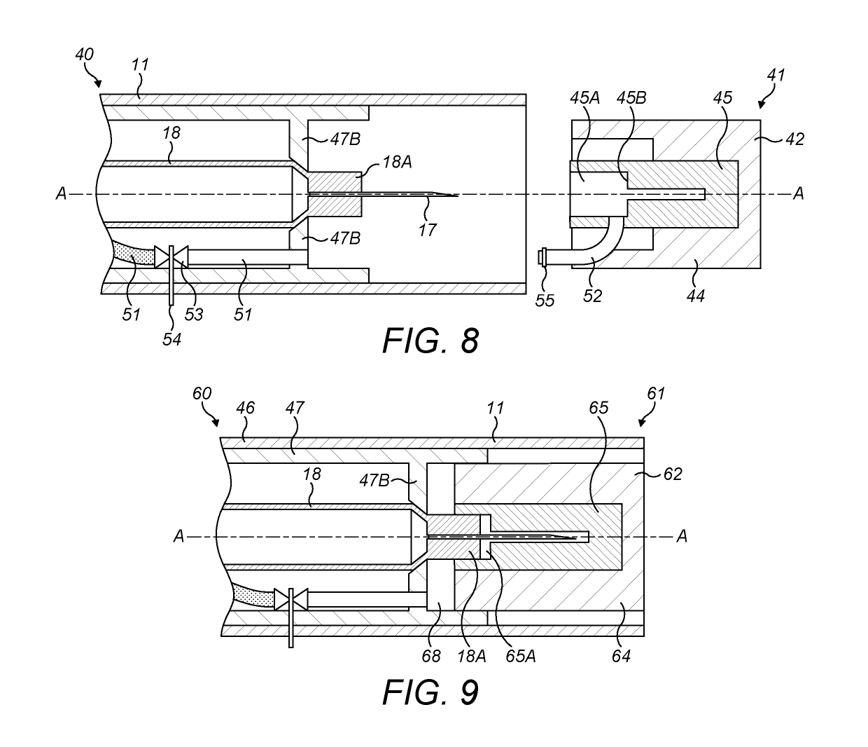

The auto-injector 40 further comprises a configuration adapted to increase the gas pressure in the chamber 49. The configuration comprises a pressurised gas source 50, first and second conduits 51, 52, a valve 53, and an actuator 54. The pressurised gas source 50 is in the form of a gas canister 50 that is disposed in the body 11 (as shown in FIG. 8). The gas canister 50 contains a gas, such as air or carbon dioxide, which is above atmospheric pressure.

The first conduit 51 is fluidly communicated with the gas canister 50 and extends to the lip 47B of the inner sleeve 47. The second conduit 52 is received in the first conduit 51 to fluidly communicate with the first conduit 51 when the cap 41 is attached to the body 11. The second conduit 52 is fluidly communicated with the chamber 49.

The actuator 54 is configured to be pressed by the patient to urge the valve 53 from a closed state (shown in FIG. 5) to an open state (shown in FIG. 7). The valve 53 is biased into the closed state. When the valve 53 is in the closed state, the valve 53 prevents gas in the gas canister 50 from flowing through the first conduit 51 to the second conduit 52.

To remove the cap 41 from the body 11, the patient presses the actuator 54 to urge the valve 53 to the open state. When the valve 53 is in the open state, pressurised gas in the gas canister 50 is able to flow through the first and second conduits 51, 52 and into the chamber 49 such that the gas pressure in the chamber 49 is increased. The increased gas pressure at the interface 48 exerts a force on the end portion 18A of the syringe 18 and on the internal surface 45B of the inner sheath 45 to move the body 11 and needle shield 42 axially apart until the cap 41 becomes separated from the body 11 (as shown in FIG. 8). The first and second conduits 51, 52 are separated when the cap 41 is separated from the body 11.

The configuration of the auto-injector 40 of the second embodiment of the invention allows for the patient to easily remove the cap 41 from the body 11. This is because the force that is required to press the actuator 54 to urge the valve 53 into the open state is less than the force which must be exerted on the cap 41 to overcome the friction that holds the cap 41 in place attached to the body 11 to separate the cap 41 from the body 11.

A filter 55 is disposed at the fluid connection between the first and second conduits 51, 52. The filter 55 removes contaminates from the gas supplied from the gas canister 50 when the valve 53 is in the open state. Therefore, the needle 17 remains sterile when the gas from the gas canister 50 enters the chamber 49. In an alternative embodiment (not shown), the filter is omitted and instead a sterile source of gas is stored in the gas canister.

Referring now to FIGS. 9 to 11, part of an injection device 60 according to a third embodiment of the invention is shown. The injection device 60 is in the form of an auto-injector 60 that has similar features to the auto-injector 40 of the second embodiment of the invention described above in relation to FIGS. 5 to 8, with like features retaining the same reference numerals. A difference is that the auto-injector 60 has an alternative cap 61 and an alternative interface 68 between the body 11 and cap 61.

The cap 61 of the auto-injector 60 of the third embodiment of the invention comprises a needle shield 62. The needle shield 62 comprises a housing 64 and an inner sheath 65. The inner sheath 65 is fixedly secured in the housing 64. The inner sheath 65 comprises a cylindrical recess 65A.

The body 11 comprises an outer casing 46 and an inner sleeve 47 that is disposed in the outer casing 46. The inner sleeve 47 is fixed relative to the outer casing 46. The needle shield 62 is received in an open peripheral end 47A of the inner sleeve 47 when the cap 61 is attached to the body 11 such that the end portion 18A of the syringe 18 is received in the recess 65A of the inner sheath 65 (as shown in FIG. 9). Therefore, the needle 17 is shielded by the inner sheath 65. The friction between housing 64 and the inner sleeve 47 and between the inner sheath 65 and the end portion 18A of the syringe 18 is sufficient to hold the needle shield 62 in place covering the needle 17.

The inner sleeve 47 comprises a lip 47B that extends radially inwardly from a peripheral wall of the inner sleeve 47 to seal against the syringe 18.

The interface 68 comprises a chamber 69 that is disposed between the proximal end 62A of the needle shield 62 and the lip 47B of the inner sleeve 47. The periphery of the housing 64 of the needle shield 62 seals against the inner surface of the inner sleeve 47 when the cap 61 is attached to the body 11 to seal the chamber 69 from atmosphere. Furthermore, the end portion 18A of the syringe 18 seals against the inner sheath 65 to seal the needle 17 from the chamber 69. Therefore, gas in the chamber 69 is prevented from contaminating the needle 17. The chamber 69 is initially at atmospheric pressure. Alternatively, the gas in the chamber 69 may initially be at a pressure that is below or above atmospheric pressure.

The auto-injector 60 further comprises a configuration adapted to increase the gas pressure in the chamber 69. The configuration comprises a pressurised gas source (not shown), a first conduit 71, a valve 73, and an actuator 74. Similarly to the auto-injector 40 of the second embodiment of the invention, the pressurised gas source of the auto-injector 60 of the third embodiment is in the form of a gas canister (not shown) that is disposed in the body 11.

The first conduit 71 is disposed in the body 11. The first conduit 71 is fluidly communicated with the gas canister and extends to the lip 47B of the inner sleeve 47.

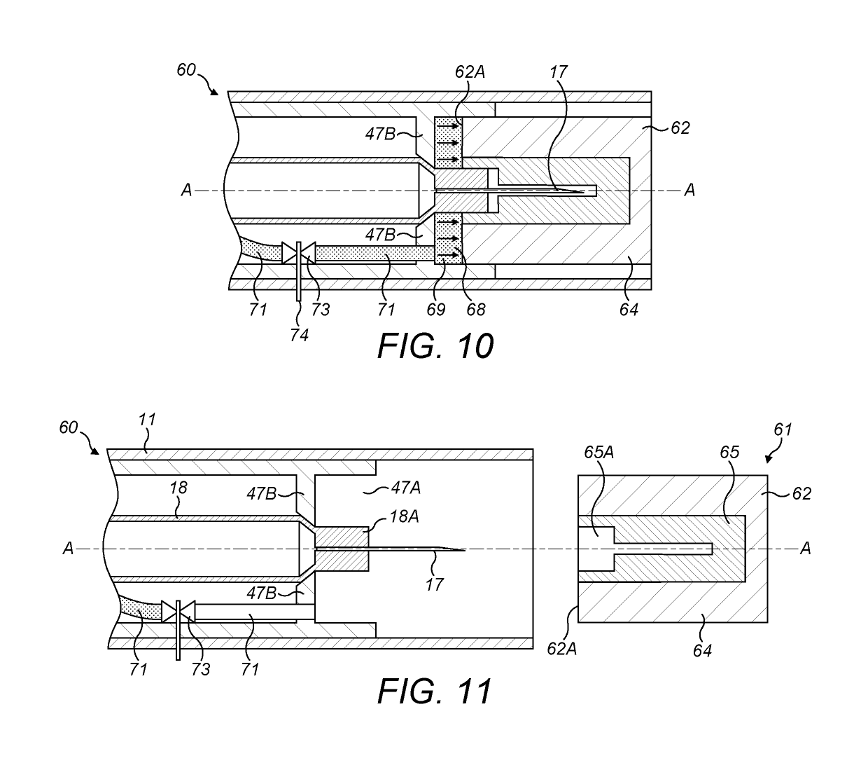

The actuator 74 is configured to be pressed by the patient to urge the valve 73 from a closed state (shown in FIG. 9) to an open state (shown in FIG. 10). The valve 73 is biased into the closed state. When the valve 73 is in the closed state, the valve 73 prevents gas in the gas canister from flowing through the first conduit 71 from the gas canister to the chamber 69.

To remove the cap 61 from the body 11, the patient presses the actuator 74 to urge the valve 73 to the open state. When the valve 73 is in the open state, the first conduit 71 fluidly communicates the gas canister with the chamber 69 such that pressurised gas in the gas canister is able to flow through the first conduit 71 and into the chamber 69 to increase the gas pressure in the chamber 69. The increased gas pressure at the interface 68 exerts a force on the proximal end 62A of the needle shield 62 and the lip 47B of the inner sleeve 47 to move the body 11 and needle shield 62 axially apart until the cap 61 becomes separated from the body 11 (as shown in FIG. 11). Therefore, in a similar manner to the auto-injector 40 of the second embodiment of the invention, the auto-injector 60 of the third embodiment of the invention allows for easy removal of the cap 61 from the body 11.

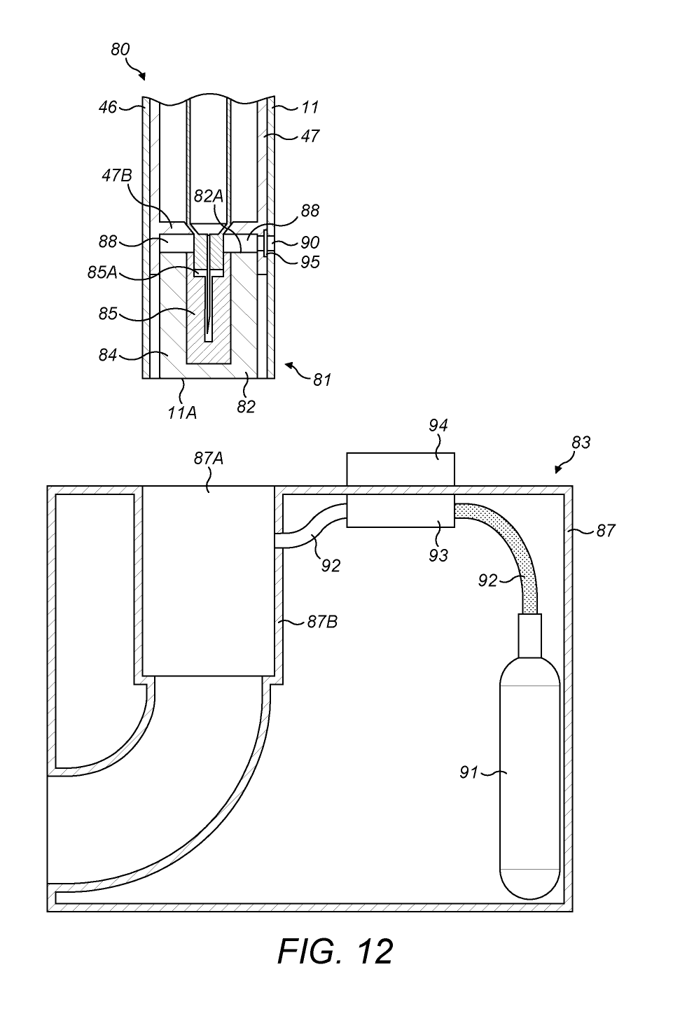

Referring now to FIGS. 12 to 16, an injection device 80 according to a fourth embodiment of the invention is shown. The injection device 80 is in the form of an auto-injector 80 that has similar features to the auto-injector 60 of the third embodiment of the invention described above in relation to FIGS. 9 to 11, with like features retaining the same reference numerals. A difference is that the auto-injector 80 of the fourth embodiment has an alternative cap 81 and further comprises a base station 83.

The cap 81 of the auto-injector 80 of the fourth embodiment of the invention comprises a needle shield 82. The needle shield 82 comprises a housing 84 and an inner sheath 85. The inner sheath 85 is fixedly secured in the housing 84. The inner sheath 85 comprises a cylindrical recess 85A.

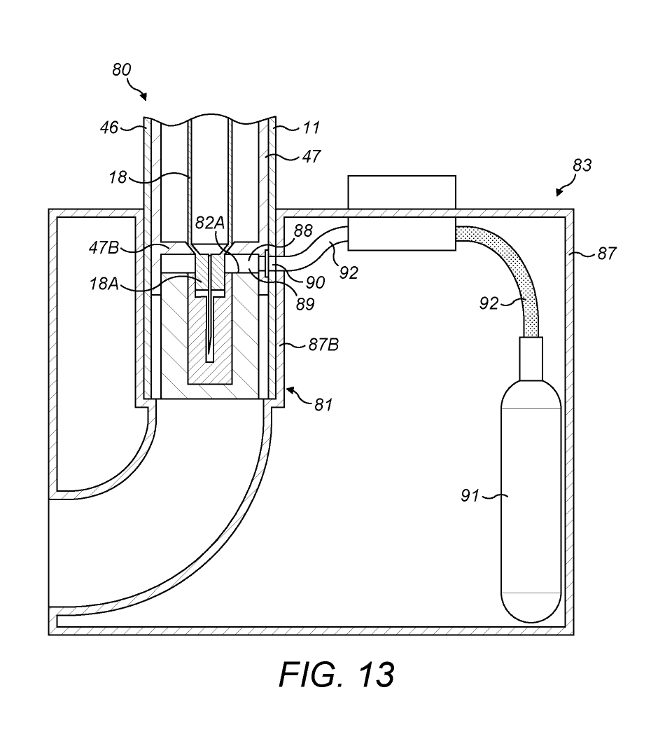

The body 11 comprises an outer casing 46 and an inner sleeve 47 that is disposed in the outer casing 46. The inner sleeve 47 is fixed relative to the outer casing 46. The needle shield 82 is received in an open peripheral end 47A of the inner sleeve 47 when the cap 81 is attached to the body 11 such that the end portion 18A of the syringe 18 is received in the recess 85A of the inner sheath 85 (as shown in FIGS. 12 and 13). Therefore, the needle 17 is shielded by the inner sheath 85. The friction between housing 84 and the inner sleeve 47 and between the inner sheath 85 and the end portion 18A of the syringe 18 is sufficient to hold the needle shield 82 in place covering the needle 17.

The inner sleeve 47 comprises a lip 47B that extends radially inwardly from a peripheral wall of the inner sleeve 47 to seal against the syringe 18.

The auto-injector 80 comprises an interface 88 that is disposed between the cap 81 and the body 11 when the cap 81 is attached to the body 11. The interface 88 comprises a chamber 89 that is disposed between the proximal end 82A of the needle shield 82 and the lip 47B of the inner sleeve 47. The periphery of the housing 84 of the needle shield 82 seals against the inner surface of the inner sleeve 47 when the cap 81 is attached to the body 11. Furthermore, the end portion 18A of the syringe 18 seals against the inner sheath 85 to seal the needle 17 from the chamber 89.

A gas-inlet 90 is provided in the peripheral wall of the body 11. The gas inlet 90 is fluidly communicated with the chamber 89 to allow for gas to flow through the peripheral wall of the body 11 and into the chamber 89. A filter 95 is provided in the gas inlet 90 to filter out any contaminates flowing into the gas chamber 89 through the gas inlet 90.

The base station 83 comprises a housing 87 with a recess 87A in the housing 87. The recess 87A is configured to receive the cap 81 and the peripheral end 11A of the body 11.

The auto-injector 80 further comprises a configuration adapted to increase the gas pressure in the chamber 89. The configuration comprises a pressurised gas source 91, a first conduit 92, a valve 93, and an actuator 94. The pressurised gas source 91 is in the form of a gas canister 91 that is disposed in the housing 87 of the base station 83 and contains a pressurised gas.

The first conduit 92 is disposed in the housing 87 of the base station 83. The first conduit 92 is fluidly communicated with the gas canister 91 and extends through a wall 87B that surrounds the recess 87A in the housing 87. Thus, the first conduit 92 fluidly communicates the gas canister 91 with the recess 87A in the housing 87.

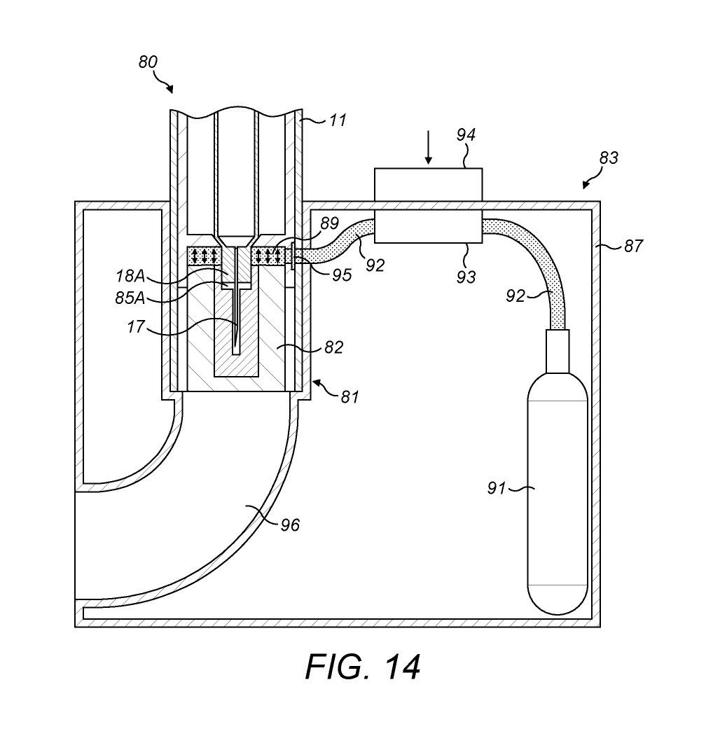

The actuator 94 is configured to be pressed by the patient to urge the valve 93 from a closed state (shown in FIGS. 12 and 13) to an open state (shown in FIG. 14). The valve 93 is biased into the closed state. When the valve 93 is in the closed state, the valve 93 prevents gas in the gas canister 91 from flowing through the first conduit 92 from the gas canister 91 to the recess 87A in the housing 87 of the base station 83.

To remove the cap 81 from the body 11, the patient first positions the cap 81 and the peripheral end 11A of the body 11 of the auto-injector 80 in the recess 87A of the housing 87 (as shown in FIG. 12). This causes the gas inlet 90 in the peripheral wall of the body 11 to be aligned with the first conduit 92 such that the chamber 89 is fluidly communicated with the first conduit 92 via the gas inlet 90.

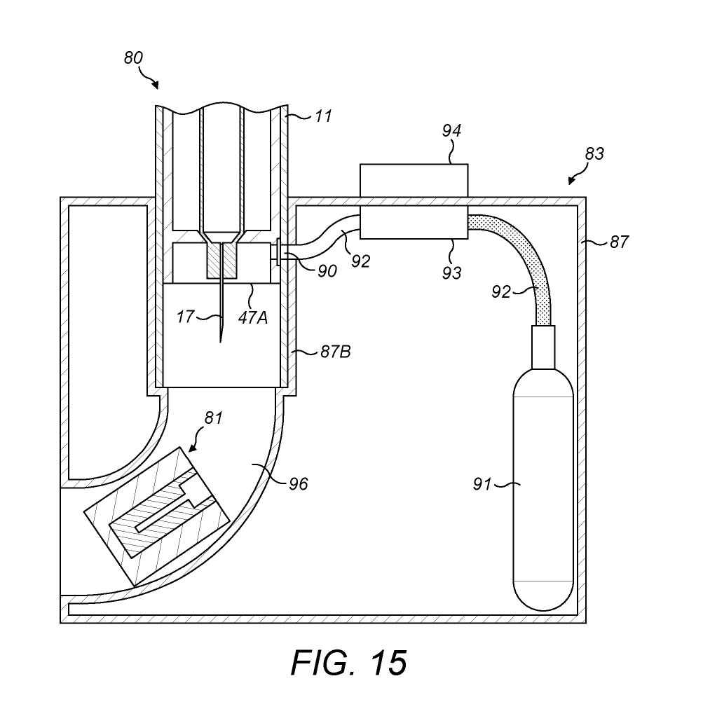

The patient then presses the actuator 94 to urge the valve 93 to the open state. When the valve 93 is in the open state, the first conduit 92 fluidly communicates the gas canister 91 with the chamber 89 such that pressurised gas in the gas canister 91 is able to flow through the first conduit 92 and into the chamber 89 to increase the gas pressure in the chamber 89. The increased gas pressure at the interface 88 exerts a force on the proximal end 82A of the needle shield 82 and the lip 47B of the inner sleeve 47 to move the body 11 and needle shield 82 axially apart until the cap 81 is separated from the body 11 (as shown in FIG. 15). Therefore, in a similar manner to the auto-injector 60 of the third embodiment of the invention, the auto-injector 80 of the fourth embodiment of the invention allows for easy removal of the cap 81 from the body 11.

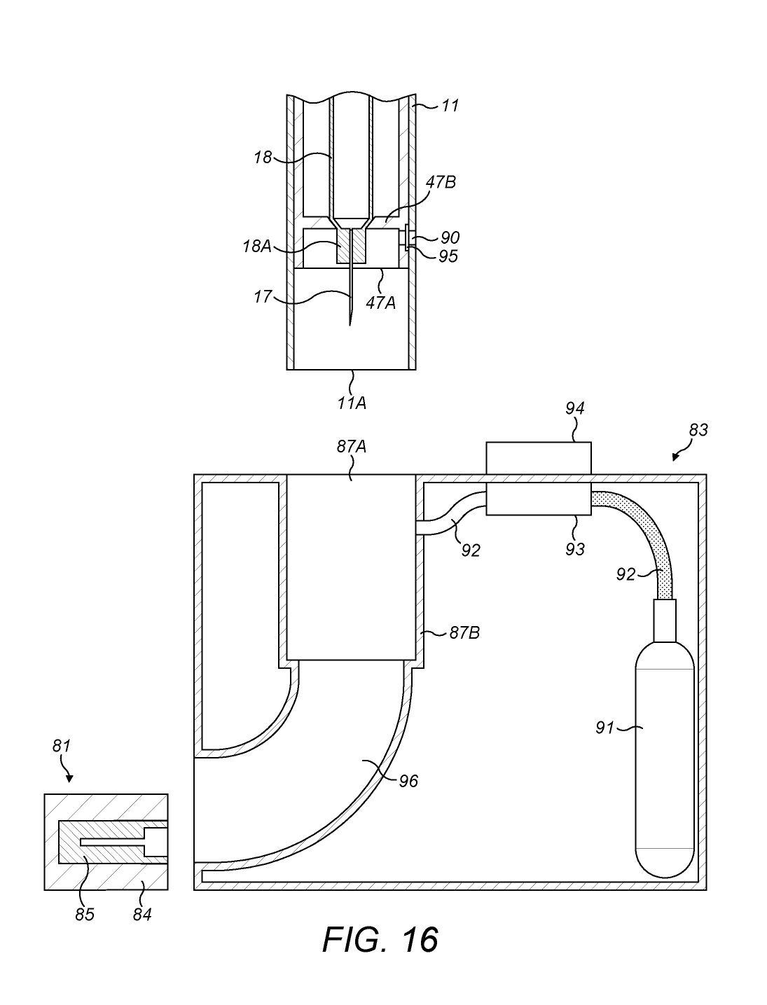

When the cap 81 has been removed from the body 11, the patient removes the body 11 from the recess 87A in the housing 87 of the base station 83 (as shown in FIG. 16). The open distal end 11A of the body 11 is then pressed up against an injection site of the patient and the dispense button (not shown) is pressed to dispense medicament to the injection site.

The base station 83 comprises a cap collection space 96 in the form of a channel 96 that extends from the recess 87A in the housing 87 to the periphery of the base station 83. The channel 96 is arranged such that when the valve 93 is urged to the open state to move the cap 81 away from the body 11 of the auto-injector 80, the cap 81 is thrust into the channel 96 by the force of the pressurised gas in the chamber 89 acting on the body 11 and cap 81. The cap 81 travels through the channel 96 and passes out of the base station 83, wherein the cap 81 can be collected by the patient and disposed of. In one embodiment, the channel 96 curves downwardly from the bottom of the recess 87A in the housing 87 and extends to a side wall of the housing 87 such that the cap 81 passes out of the side wall when the cap 81 is removed from the body 11 (as shown in FIG. 16).

In an alternative embodiment (not shown), the cap collection space instead comprises a collection chamber inside the housing of the base station. The collection chamber extends from the recess in the housing and is configured such that the cap moves into the collection chamber when the valve is urged to the open state to remove the cap from the body. The removed cap is stored in the collection chamber until subsequent disposal by the user. In one such embodiment, the collection chamber is sufficiently large to store a plurality of removed caps.

In the above described embodiment the pressurised gas source 91 is in the form of a gas canister. However, in an alternative embodiment (not shown), the injection device instead comprises a flexible portion which defines a cavity that forms a deformable space. The deformable space is fluidly communicated with the interface, for example by a conduit, and is sealed from the atmosphere when the cap is attached to the body. The flexible portion is squeezed to increase the pressure at the interface such that the needle shield and body are urged apart. The flexible portion may form part of the cap, the body, or the base station. In yet another embodiment (not shown), the injection device instead comprises an actuator that is operable by the user to compress a space that is fluidly communicated with the interface. Thus, when the user operates the actuator the pressure in the space is increased to urge the needle shield and body apart. The actuator may be slidably coupled to the cap, body or base station.

In the above described embodiments, the cap 21, 41, 61, 81 and body 11 are both generally cylindrical. However, is should be recognised that injection devices having caps and bodies which are shapes other than cylindrical intended to fall within the scope of the disclosure. For example, the cap and body may instead have a square, oval, rectangular, pentagonal or hexagonal shape when viewed in cross-section.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injection devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(ω-carboxyheptadecanoyl)-des(B30) human insulin and B29-N-(ω-carboxyhepta¬decanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in certain aspects of the present invention include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

1-15. (canceled)

16. An injection device comprising:
a body for holding a syringe that has a needle at one end;
a cap that is removably attached to the body and that comprises a needle shield to cover the needle; and
a pressurization mechanism adapted to increase gas pressure at an interface of the injection device that is disposed between the body and a portion of the cap to urge the body and the portion of the cap apart.

17. The injection device according to claim 16, wherein the interface comprises a chamber configured to be disposed between the body and the portion of the cap when the cap is attached to the body.

18. The injection device according to claim 17, wherein the chamber is configured to be disposed between the syringe and the portion of the cap when the cap is attached to the body and when the syringe is received in the body.

19. The injection device according to claim 16, wherein the needle shield comprises the portion of the cap.

20. The injection device according to claim 16, wherein the pressurization mechanism comprises a compressed gas source configured to be selectively in fluid communication with the interface.

21. The injection device according to claim 16, further comprising a base station that has a housing with a recess to receive the cap.

22. The injection device according to claim 21, wherein the base station comprises a cap collection space configured such that the cap moves into the cap collection space when the cap is received in the recess of the housing and when the pressurization mechanism urges the portion of the cap away from the body.

23. The injection device according to claim 16, wherein the pressurization mechanism comprises a space that is in fluid communication with the interface and is compressible to increase the gas pressure at the interface.

24. The injection device according to claim 23, wherein the space is disposed in one of the body or cap, and wherein at least a part of the one of the body or cap is deformable to compress the space.

25. The injection device according to claim 23, wherein the space is disposed in one of the body or cap, and wherein the one of the body or cap comprises an actuator that is slidable relative to the other one of the body or cap to compress the space.

26. The injection device according to claim 25, wherein the injection device is configured such that a force exerted on the actuator to slide the actuator relative to the body to compress the space results in a larger force being exerted to urge the body and the portion of the cap apart.

27. The injection device according to claim 16, comprising a syringe having a needle at one end and being received in the body, wherein the needle shield is in frictional engagement with the syringe.

28. The injection device according to claim 16, comprising a syringe that has a needle at one end, wherein the syringe contains a medicament.

29. The injection device according to claim 16, wherein the injection device is an auto-injector.

30. The injection device according to claim 16, wherein the pressurization mechanism comprises an actuator movable toward the body of the injection device to increase the pressure at the interface.

31. The injection device according to claim 30, wherein the actuator is movable proximally toward the body of the injection device to increase the gas pressure at the interface.

32. A method comprising:
removing a cap from a body of an injection device by increasing a gas pressure at an interface disposed between the body and a portion of the cap to urge the body and the portion of the cap apart.

33. The method of claim 32, further comprising, before removing the cap, releasing a lock that prevents the cap from moving axially relative to the body.

34. The method of claim 32, wherein removing the cap comprises moving an actuator axially toward the body of the injection device, thereby causing the cap to move axially away from the body.

35. The method of claim 34, wherein moving the actuator axially toward the body of the injection device comprises moving the actuator proximally toward the body of the injection device.
